(19) United States
(12) Plant Patent Application Publication
(10) Pub. No.: US 2007/0204371 P1
(43) Pub. Date: Aug. 30, 2007

(54) ARGYRANTHEMUM FRUTESCENS PLANT NAMED 'ARGYMINPIFI'

(75) Inventor: Anna M.W.P. Houbraken, Enkhuizen (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/361,093

(22) Filed: Feb. 24, 2006

(51) Int. Cl. A01H 5/00

(52) U.S. Cl. PLT/263

(57) ABSTRACT

A new and distinct cultivar of Argyranthemum frutescens plant, substantially as herein illustrated and described, characterized particularly as to novelty by its compact rounded plant habit, good field performance, dark green colored leaves, freely flowering with numerous inflorescences per plant, and daisy-type inflorescences with pink double flowered inflorescences, organized in a crumpled way.

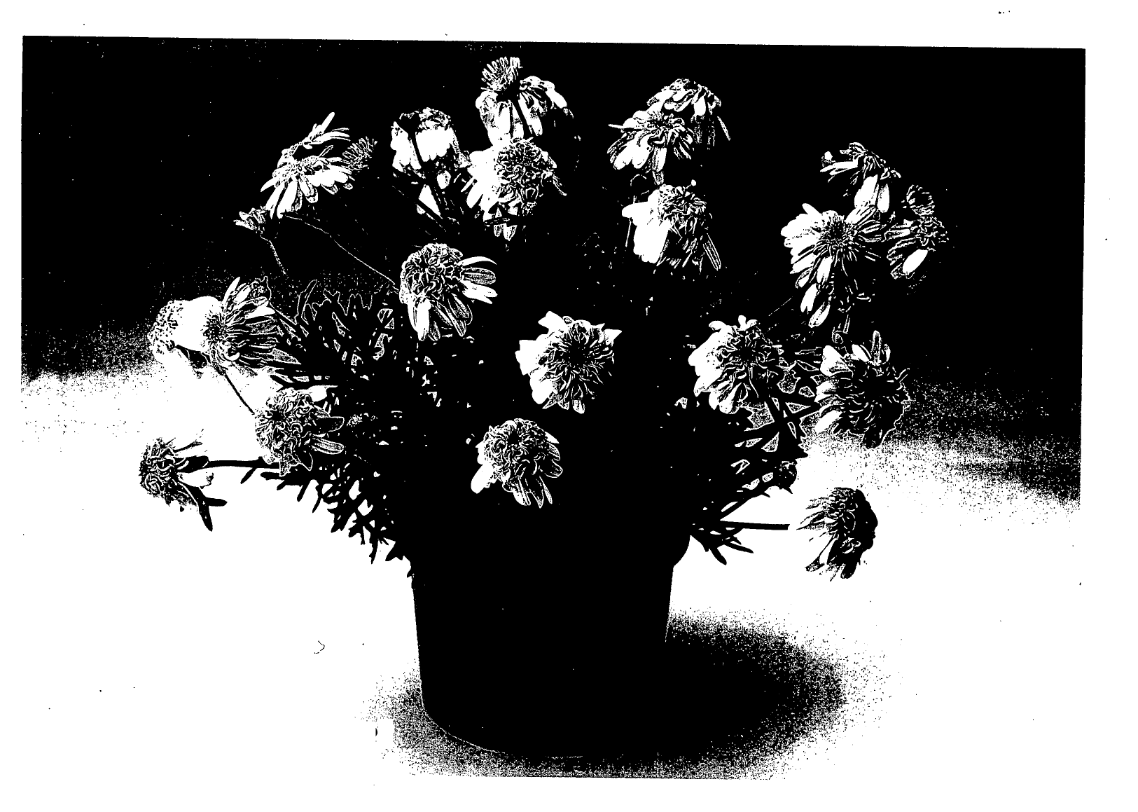

### LATIN NAME OF THE GENUS AND SPECIES OF THE PLANT CLAIMED

Argyranthemum frutescens

### VARIETAL DENOMINATION ‘Argyminpifi’

### BACKGROUND OF THE NEW PLANT

The present invention relates to a new and distinct cultivar of Marguerite Daisy plant, botanically known as Argyranthemum frutescens and hereinafter referred to by the cultivar name ‘Argyminpifi.’

The new cultivar is a product of a planned breeding program conducted by the inventor in Enkhuizen, The Netherlands. The objective of the breeding program was to create new, compact Argyranthemums with early freely flowering habit, which can be grown without growth regulators.

The new Argyranthemum originated from an open pollination conducted in Enkhuizen, The Netherlands in 2000 of a proprietary selection of Argyranthemum frutescens identified as code number ‘D0107,’ not patented, as the female, or seed, parent with an unknown selection of Argyranthemum frutescens as the male, or pollen, parent.

The cultivar ‘Argyminpifi’ was discovered and selected as a single plant within the progeny of the stated open pollination in a controlled environment in Enkhuizen, The Netherlands in August 2001.

Asexual reproduction of the new Argyranthemum by terminal cuttings in a controlled environment in Enkhuizen, The Netherlands since August 2001, has shown that the unique features of this new Argyranthemum are stable and reproduced true to type in successive generations.

### SUMMARY OF THE INVENTION

The new Argyranthemum has not been observed under all possible environmental conditions. The phenotype may vary somewhat with variations in environment such as temperature and light intensity without, however, any variance in genotype.

The following characteristics have been repeatedly observed and are determined to be basic characteristics of ‘Argyminpifi’ and distinguish to new Argyranthemum as a new and distinct cultivar:

1. Compact, upright and rounded plant habit

2. Freely branching habit and short internodes; dense and bushy plant habit.

3. Early flowering habit

4. Freely flowering habit with numerous inflorescences per plant.

5. Daisy-type inflorescences with pink ray florets in double inflorescences.

Plants of the new Argyranthemum can be compared to plants of the cultivar ‘Summer Melody,’ ‘Argywhimi’ and ‘Summer Stars Pink.’ In side-by-side comparisons conducted in Enkhuizen, The Netherlands, plants of the new Argyranthemum differed from plants of the comparison varieties in the following characteristics:

TABLE 1
Differences between the new cultivar ‘Argyminpifi’ and
three similar cultivars

| | | ‘Summer | ‘Summer | |
|---|---|---|---|---|
| | ‘Argyminpifi’ | Melody’ | Stars pink’ | ‘Argywhimi’ |
| Plant | 25 cm | 26 cm | 50 cm | 26 cm |
| Height | | | | |
| Flower | Double | Double | Double | Single |
| shape | crumpled | | crumpled | |
| | frizzly | | frizzly | |
| Flower | Pink | Rose | Pink | White |
| color | | | | |

### DESCRIPTION OF THE DRAWING

The accompanying colored photographs illustrate the overall appearance of the new cultivar, showing the colors as true as it is reasonably possible to obtain in colored reproductions of this type. Colors in the photograph may differ slightly from the color values cited in the detailed botanical description which accurately describe the colors of the new Argyranthemum.

### DESCRIPTION OF THE NEW CULTIVAR

The aforementioned photographs, following detailed observations and averaged measurements describe plants of this new Marguerite Daisy plant. The data were collected from plants from asexual reproductions carried out in Enkhuizen, The Netherlands. The plant history was taken on 30 week old plants, grown in the open field in Enkhuizen, The Netherlands with day temperatures ranging from 16 to 30 degrees C., and night temperatures ranging from 10 to 16 degrees C.

Color references are primarily to the RHS Colour Chart of the Royal Horticultural Society, 1995 Edition, except where general terms of ordinary dictionary significance are used.

### The Plant

Classification
   Botanical.—Argyranthemum frutescens cv. ‘Argyminpifi’.

### Parentage

Female parent: Proprietary seedling section of Argyranthemum frutescens, identified as number ‘D0107’ (not patented). Breeder's code of the new plant is ‘E0155-1.’
Male parent: Unidentified section of Argyranthemum frutescens (not patented)

### Propagation

Type cuttings: Terminal vegetative cuttings
Time to initiate roots: 7-10 days at air temperature of 21 degrees C.
Time to develop roots: 7-14 days at air temperature of 18 degrees C.
Root description: Fibrous, relatively fine, white in color
Rooting description: Freely branching, dense

### Plant Description

Growth habit: Compact and rounded shape
Plant height: About 25 cm
Vigor: Compact, less vigorous
Spreading area of plant: About 30-35 cm
Strength: Very good
Branching character: Freely branching, plants do not require pinching
Crop time: About 9 weeks are required to produce a finished flowering plant in a 10.5 cm container from a good developed cutting

### Lateral Branch Description

Length: 14cm
Diameter: 4 mm
Shape: Round, bit grooved
Anthocyanin pigmentation: Absent
Texture: Smooth, glabrous
Length of internode: 0.5-1.5 cm
Pubescence: Absent
Color: 144A

### The Foilage

Arrangement: Alternate, simple
Shape of leaf: Bipinnatisect
Leaf apex: Broadly acute
Leaf base: Attenuate, clasping
Margin: Deeply dissected, serrated
Texture: Thick glabrous smooth
Length: 3-4 cm
Width 1-2 cm
Depth of incision: Lanciniate
Color:
   Upper side.—137A.
   Lower side.—137A.
   Old leaf.—137A.
Pubescence: Absent
Petiole:
   Length.—About 0.5-1 cm.
   Diameter.—About 2 mm.
   Texture.—Smooth glabrous.
   Color.—137B.
Venation:
   Shape.—Pinnate.
   Color.—137A.

### Inflorescence Description

Appearance: Daisy-type inflorescences with ligulate ray florets. Disc and ray florets develop acropetally on a capitulum. Inflorescences held upright and outwardly on terminal and axillary peduncles. Inflorescences positioned perpendicular to the peduncles. Inflorescences persistent.
Flowering response: Under natural conditions, plants flower from spring to early fall in N-W Europe; plants flower continuous during this period.
Inflorescence longevity: Inflorescences last about twelve days on the plant
Quantity of inflorescences: Freely flowering, about 200 open inflorescences and inflorescence buds per plant

### Flower Bud

Peduncle:
   Length.—3.5-4 cm.
   Diameter.—1 mm.
   Strength.—Strong.
   Aspect.—Upright to slightly outward.
   Anthocyan.—Present.
   Texture.—Smooth, glabrous.
   Color.—144A.
Inflorescence bud:
   Height.—3-5 mm.
   Diameter.—5-7 mm.
   Shape.—Round.
   Color.—161B.

### Inflorescence

Size:
   Diameter.—3-3.5 cm.
   Depth.—About 1 cm.
Diameter of disc: About 3 mm
Form: Double flowered with ray florets crumpled (frizzly)
Receptacle:
   Height.—About 5 mm.
   Diameter.—About 9 mm.
Involucre/phyllaries: 3 series of 5 bracts, tightly to receptacle
   Appearance.—Margins, papery and membranous.
   Shape.—Elliptic.
   Apex.—Broad.
   Base.—Truncate.
   Texture.—Smooth, glabrous.
   Margin.—Entire.
   Length.—3 mm.
   Width.—1-2mm.
   Color.—Upper: 144A; Lower: 139B.
Fragrance: Absent

### Ray Florets

Shape of ray florets: Ligulate
Apex: Emarginate
Base: Attenuate
Margin: Entire
Color:
   Upper surface.—75D.
   Lower surface.—75D.
No. of ray florets per inflorescence: 25 arranged in each 6-8 whorls, organized frizzly
Size outer ray florets: Length: 12 mm; Width: 4 mm
Size inner ray florets: Length: 6 mm, Width: 4 mm
Texture: Smooth, glabrous, longitudinally ridged

### Disc Florets

Arrangement: Massed at the center of the inflorescence
Shape of disc florets: Tubular, elongated, five-pointed, base, fused
Color:
   Immature.—68B; tip is N57B.
   Mature.—14B.
Diameter of disc floret:
   Apex.—1mm.
   Base.—<1 mm.
Length of disc floret: 2 mm
Quantity per inflorescence: Numerous, ±50

### Androecium

Present only on disc florets
Stamen quantity per floret: Five
Anther length: About 1 mm
Anther shape: Oval
Anther color: Yellow 21 A
Pollen color: Not observed
Amount of pollen: Not observed

### Gynoecium

Present on ray and disc florets
Pistil number: One per floret
Pistil length: ±3 mm
Style color: Yellow green 144D
Style length: ±1 mm
Stigma color Yellow orange 14A
Stigma shape: Bilobed
Ovary color: 144C

### Seed/fruit

Seed and fruit production is not observed.

### Disease Resistance

No susceptibility nor resistance to fungal, bacterial or viral pathogens has been noted. Material is free of virus and Chrysanthemum stunt void (CSVd).

### Temperature/Weather Tolerance

Plants of the new Argyranthemum have been observed to be tolerant to rain, wind and to temperatures ranging from 0 to 36 degrees C.

What is claimed is:

1. A new and distinct variety of Argyranthemum frutescens plant named ‘Argyminpifi,’ as substantially illustrated and described herein.
